(19) United States
(12) Patent Application Publication
(10) Pub. No.: US 2008/0118980 A1
(43) Pub. Date: May 22, 2008

(54) Estrogen Enhancing Differentiation of Human Mesenchymal Stromal Cells and Adipose-derived Stromal Cells

(75) Inventor: Liu Hong, Palos Hills, IL (US)

Correspondence Address: GREENLEE WINNER AND SULLIVAN P C, 4875 PEARL EAST CIRCLE, SUITE 200, BOULDER, CO, 80301, US

(73) Assignees: The Board of Trustees of the University of Illinois,; a body corporate and politic of the State of Illinois, Urbana, IL (US)

(21) Appl. No.: 11/855,938

(22) Filed: Sep. 14, 2007

Related U.S. Application Data
(60) Provisional application No. 60/825,700, filed on Sep. 14, 2006.
(60) Provisional application No. 60/869,061, filed on Dec. 7, 2006.

(51) Int. Cl. C12N 5/06

(52) U.S. Cl. 435/377

(57) ABSTRACT

Adipose-derived stromal cells and bone marrow mesenchymal stem cells possess multiple differentiation potentials and can provide tissues for regenerative medicine and tissue engineering. The present invention provides a method of enhancing the differentiation and proliferation of adult stem cells, such as adipose-derived stromal cells and bone marrow mesenchymal stem cells, by contacting the adult stem cells with an effective amount of one or more estrogen receptor ligands. In particular, the present invention provides methods of enhancing osteogenesis and adipogenesis by contacting adipose-derived stromal cells and bone marrow mesenchymal stem cells with 17-β estradiol.

### CROSS-REFERENCE TO RELATED APPLICATIONS

This application claims the benefit of U.S. Provisional Application 60/825,700, filed Sep. 14, 2006 and U.S. Provisional Application 60/869,061, filed Dec. 7, 2006. Both applications are incorporated herein in their entirety to the extent not inconsistent herewith.

### BACKGROUND OF THE INVENTION

Annually, hundreds of thousands of patients suffer from various diseases or traumas resulting in hard or soft tissue defects requiring regeneration or restoration. Adult stem cell based tissue engineering and regeneration characterizing high efficiency, safety, and ethical considerations, has been proposed as a promising substitute for restoration of such tissue defects. (Mauney et al. (2005) Tissue Eng 11, 787-802; Tuan, R. S. (2004), Clin Orthop Relat Res 427 Suppl., S105-S117; Cancedda et al. (2003) Stem Cells 21, 610-619.)

Adipose-derived stromal cells (ASCs) and bone marrow mesenchymal stem cells (MSCs) possess multiple differentiation potentials and are capable of osteogenic, adipogenic, myogenic, and chondrogenic differentiation. Thus, ACSs and MSCs may serve as a cell source, if effectively modulated, for the different tissue used in regenerative medicine and tissue engineering to restore bone and other tissue defects caused by trauma, tumor dissection, and congenital insufficiency. However, as cell sources for tissue engineering, multipotent MSCs and ASCs have disadvantages including insufficient supply and low differentiation potentials.

For example, MSCs isolated from bone marrow are capable of osteoblastic, adipocytic, chondrocytic, and myoblastic differentiation and are considered a cell source for stem cell-based tissue engineering (Caplan, Al. (2005) Tissue Eng. 2005; 11:1198-1211; Kassem, M. (2004) Cloning Stem Cells 6:369-374; Barry et al. (2004) Int J Biochem Cell Biol. 36:568-584). However, the population of MSCs in bone marrow is often too low to satisfy the requirement of tissue regeneration and several patho-physiological conditions can directly influence MSC numbers and differentiation characteristics (Aubin J E et al. (2002) Principles of bone biology, Second edition, Academic Press; Caplan et al. (1998) Adv Drug Deliv Rev. 33:3-14). In addition, the in vitro MSC expansion capability and differentiation characteristics are donor-dependant. Further, in vitro MSCs' proliferation rate, differentiation potentials and capacity for tissue formation significantly decrease over time (Jaiswal et al. (1997) J Cell Biochem. 64:295-312).

Ubiquitous adipose tissue develops from mesoderm, similar to bone marrow tissue, and has been shown to contain progenitor cells with multiple differentiation potentials, called adipose-derived stem or stromal cells (ASCs). ASCs possess several advantages over bone marrow MSCs, including sufficient supply with minimally invasive procurement. Under specific differentiation conditions, similar to bone marrow MSCs, ASCs can also differentiate into osteogenic, adipogenic, myogenic, chondrogenic, neurogenic cell lineages (Zuk et al (2001) Tissue Eng. 7:211-228; Cowan et al. (2004) Nat Biotechnol. 22:560-567). However, the capacity for multiple differentiations of ASCs is generally lower than bone marrow MSCs (Huang et al. (2005) J Orthop Res. 23:1383-1389; De Ugarte et al. (2003) Cells Tissues Organs. 174:101-109). Previous studies have suggested that effective modulation to improve differentiation potentials may permit the use of ASCs as a cell source for cell-based tissue engineering (Cho et al. (2005) J Cell Biochem. 96:533-542; Dragoo et al. (2003) J Orthop Res. 21:622-629; Knippenberg et al. (2005) Tissue Eng. 11:1780-1788). What is needed is a way to increase the number of available adult stem cells, particularly both ASCs and MSCs, and enhance the differentiation potential of these cells.

The estrogen receptor (ER) is a member of the nuclear hormone receptor superfamily, which mediates the activity of estrogens in the regulation of a number of important physiological processes, including the development and function of the female reproductive system and the maintenance of bone density and cardiovascular health. Estrogen has multifunctional roles influencing growth, differentiation and metabolism in many tissues. Estrogen exerts its functions via its receptors that exist in multiple progenitor cells of bone marrow and adipose tissue.

It had been assumed that estrogen-related events were mediated by only one estrogen receptor. However, the discovery of a second estrogen receptor (ER beta) (Mosselman, et al. (1996) FEBS Lett., 392, 49-53; Kuiper et al. (1996) Proc. Natl. Acad. Sci. USA, 93, 5925-5930) indicates that tissue- and cell-selectivity of certain estrogens may be due, in part, to their mediation through ER beta separate from, or in conjunction with, the classical estrogen receptor (ER alpha). This possibility has been supported by the difference in tissue distribution between ER alpha and ER beta (Mosselman et al. (1996) FEBS Lett. 392, 49-53; Kuiper et al. (1997) Endocrinology 138:863-870; Saunders et al. (1997) J. Endocrinol. 154:R13-R16; Register et al. (1998) J. Steroid Biochem. Mol. Biol. 64:187-191.)

ER alpha and ER beta (also referred to herein as ERα and ERβ) exhibit complex tissue distributions. Certain tissues may contain only (or predominately) ER alpha or ER beta and other tissues may contain a mixture of both ER alpha and ER beta. Tissues that exhibit high levels of ER beta include, for example, prostate, testes, ovaries, gastrointestinal tract, lung, bladder, hematopoetic and central nervous systems, and certain regions of the brain, whereas ER alpha predominates in the uterus, breast, kidney, liver and heart. Tissues that exhibit both ER alpha and ER beta include breast, epididymis, thyroid, adrenal, bone, and certain other regions of the brain. Furthermore, it has been shown that the pharmacology of traditional ER agonists and antagonists is reversed for ER beta in the context of certain ER effector sites. (Paech et al. (1997) Science 277:1508-1510.)

The primary ER ligands are estriol (also referred to herein as E3), estradiol, and estrone, particularly 17-β estradiol; however, foreign ligands, including artificial ligands, can be introduced into cells to serve as ER agonists. 17-β estradiol (also referred to herein as E2) has been shown to affect bone, cartilage, and adipose tissues (Heim et al. (2004) Endocrinology. 145:848-859; Dang et al. (2004) J Bone Miner Res. 19:853-861; Cooke et al. (2004) Exp Biol Med (Maywood) 229:1127-1135; Talwar et al. (2006) J Oral Maxillofac Surg. 64:600-609). Although the two ER subtypes are both activated by binding estradiol (E2), the ligand binding domain (LBD) and activation function-2 (AF-2) region of the subtypes is only 56% conserved and the A/B domain/activation function-1 (AF-1) is only 20% conserved (Mosselman et al. (1996) FEBS Lett. 392, 49-53; Kuiper et al. (1996) Proc. Natl. Acad. Sci. USA, 93, 5925-5930). Ligands may be developed which bind to the two ER subtypes differently and perhaps have different affinities, potencies, and agonist vs. antagonist character. Ligands may be developed which selectively activate only one ER subtype, thereby mediating cell or tissue-selective gene expression and/or mediating beneficial estrogenic effects and minimizing detrimental estrogenic effects.

### SUMMARY OF THE INVENTION

The present invention provides a method of proliferating and enhancing the differentiation of adult stem cells by contacting the cells with estrogen receptor ligands. Adult stem cells are capable of forming several different cell types, including but not limited to osteogenic, adipogenic, myogenic, chondrogenic, neurogenic formation. Typically the end cell type is determined by the medium added to the undifferentiated adult stem cells. Solutions capable of directing the differentiation of adult stem cells into specific cell types are well known in the art. For example, an osteogenic medium containing dexamethasone, β-glycerophosphate, and ascorbic acid-2-phosphate is added to undifferentiated adult stem cells to cause osteogenic differentiation, and an adipogenic medium containing dexamethasone, insulin and isobutyl-methylxanthine (IBMX) is added to cause adipogenic differentiation. Supplementing the mediums with an estrogen receptor ligand increases the purity and number of the resulting differentiated cells. Due to estrogen's function in tissue and organ development through regulating cell proliferation and differentiation, it is believed that an estrogen supplement may effectively enhance the multiple differentiation potentials of human adult stem cells such as ASCs and MSCs. One embodiment of the present invention provides a method of enhancing the differentiation of adult stem cells by isolating the cells, contacting the isolated cells with a solution to determine differentiation, and contacting the cells with an effective amount of one or more estrogen receptor ligands. The estrogen receptor ligands may be added simultaneously with the differentiation determining solution or added to the cells soon after the differentiation determining solution has been added.

The adult stem cells suitable for use with the present invention include, but are not limited to, adipose-derived stromal cells (ASCs) and bone marrow mesenchymal stem cells (MSCs). In one embodiment, one or more estrogen receptor ligands are used to enhance the osteogenic and adipogenic differentiation of ASCs and MSCs. Preferably, the one or more estrogen receptors are selected from the group consisting of estriol, estradiol, estrone and combinations thereof, more preferably 17-β estradiol. In a further embodiment, between about 10⁻⁴ M and about 10⁻¹⁴ M of the estrogen receptor ligand is administered to the cells, more preferably between about 10⁻⁶ M and about 10⁻¹² M; even more preferably between about 10⁻⁸ and 10⁻¹¹ M or between about 10⁻⁶ and about 10⁻⁹ M.

One embodiment of the invention provides a method for improving the osteogenic differentiation of ASCs and MSCs by contacting isolated ASCs or MSCs with an osteogenic medium supplemented with a first effective amount of one or more estrogen receptor ligands. The effectiveness of osteogenic differentiation can be determined through the measurement of certain osteogenic markers. Increased alkaline phosphatase (ALP) activity, core binding factor alpha1 (cbfa1), extracellular matrix calcium deposition, and osteocalcin (OCN) expression all indicate increased osteogenic differentiation.

Another embodiment of the present invention provides a method for improving the adipogenic differentiation of ASCs and MSCs by contacting isolated ASCs or MSCs with an adipogenic medium supplemented with a first effective amount of one or more estrogen receptor ligands. The effectiveness of adipogenic differentiation can be determined through measurement of certain adipogenic parameters (such as lipid accumulation, differentiated cell population, and levels of peroxisome proliferator activated receptors gamma (PPARγ)-2).

In another aspect of the present invention, the proliferation of adult stem cells in culture is increased by contacting isolated adult stem cells with estrogen receptor ligands. In one embodiment, undifferentiated adult stem cells are cultured in standard growth medium supplemented with a second effective amount of one or more estrogen receptor ligands. The estrogen receptor ligands increase proliferation of the adult stem cells without adversely affecting the ability of the stem cells to differentiate. This method can be used to increase the number of adult stem cells independent of subsequent differentiation steps, or this method can be used in conjunction with other methods of the invention to increase the number of adult stem cells followed by enhancement of the differentiation of the adult stem cells. Preferably, the estrogen receptor ligands comprise estriol (E3), 17-β estradiol (E2), or both. Preferably between about 10⁻⁶ M and about 10⁻¹² M of the estrogen receptor ligand is administered to the adult stem cells to increase proliferation; even more preferably between about 10⁻⁶ M and about 10⁻⁹ M. The amount of estrogen receptor ligands used to increase proliferation of the cells may be the same or different than the amount used to subsequently enhance differentiation.

The number and distribution of estrogen receptors may differ between male cells and female cells (i.e., cells coming from a male member or female member of the species) depending on the particular species and cell type. This indicates that the strength of the proliferation and differentiation effects of estrogen receptor ligands may differ in male cells and female cells. For example, female cells may generally exhibit a stronger response to estrogen receptor ligands than male cells. Additionally, the optimal concentration of estrogen receptor ligands for regulatory function of proliferation and differentiation may vary with genders. In a further embodiment of the invention, the concentration of estrogen receptor ligands administered to adult stem cells is adjusted according to the gender of the cell. For example, the optimal estrogen receptor ligand concentration may adjusted to about 10⁻⁶ M for female cells and to about 10⁻⁹ M for male cells for proliferation and osteogenic or adipogenic differentiation of adult stem cells.

### BRIEF DESCRIPTION OF THE DRAWINGS

FIG. 1 shows RT-PCR results of osteogenic differentiation of human bone marrow MSCs (left) and ASCs (right) modulated by E2 with different concentrations.

FIG. 2 shows quantitative measurement of estrogen's modulations on osteogenic differentiation of MSCs and ASCs with different concentrations. DNA contents (A:MSCs; D: ASCs), ALP activities (B:MSCs; E: ASCs) and calcium contents (C:MSCs; F: ASCs) of osteogenic differentiated MSCs and ASCs. p<0.05.

FIG. 3 shows multiple differentiation potentials of human ASCs. A: osteogenic nodules marked with active ALP and mineral deposition were formed after 14 days of exposure to osteogenic differentiation medium (ALP and von Kossa staining). C: adipogenic differentiated ASCs characterized by lipid accumulation were observed after 7 days of exposure to adipogenic differentiation (Oil-O-Red staining). B: (ALP and von Kossa staining) and D: (Oil-O-red staining) are ASCs cultured in the basic medium after 14 and 7 days, respectively. Bar=100 μm.

FIG. 4 shows RT-PCR results of osteogenic differentiation of human ASCs modulated by 17β-estradiol after exposure to 7 days of differentiation medium.

FIG. 5 quantitative measurement of osteogenic differentiation of ASCs improved with different concentrations of E2 supplement. DNA contents (A), ALP activity (B), protein content (C), and calcium content (D) of osteogenic differentiated ASCs. *: p<0.05; **: p<0.01. N=4.

FIG. 6 shows RT-PCR results of adipogenic differentiation of human ASCs after exposure to 4 days of 17β-estradiol at different concentrations.

FIG. 7 shows quantitative measurement of adipogenic differentiation of ASCs after exposure to 7 days of E2 supplement at different concentrations. DNA contents (A), adipocyte number (B) and elution of lipid accumulation (C) of adipogenic differentiated ASCs. *: p<0.05; **: p<0.01. N=4.

FIG. 8 shows results of cell proliferation on MSC cultures supplemented with 1000 nM and 10 nM of E2 and E3.

FIG. 9A shows the effect of E2 on the cell proliferation of male and female MSCs. FIG. 9B shows ALP activity for osteogenic differentiation of male and female MSCs treated with various concentrations of E2. FIG. 9C shows calcium content for osteogenic differentiation of male and female MSCs treated with various concentrations of E2.

### DETAILED DESCRIPTION

ER ligands are any compounds which exhibit measurable binding affinity for the estrogen receptor. The primary ER ligands are estriol, estradiol, and estrone, particularly 17-β estradiol (E2). There are various ways to measure and quantify ER binding affinity. Binding affinity is expressed as a relative binding affinity (RBA) in percent compared to estradiol which is assigned an affinity of 100%. Substantial affinity for ER is indicated by an RBA of about 0.1% or more. Good affinity binding to ER is indicated by an RBA of about 1% to about 10%. High affinity binding to ER is indicated by an RBA of about 10% or higher. When binding affinity for ER subtypes is measured, binding affinity is expressed as an RBA in percent relative to the affinity of estradiol for the ER subtype which is assigned an affinity of 100%. ER ligands include naturally occurring compounds administered to cells in amounts that are not usually present in the body. ER ligands also include artificially synthesized compounds.

An “effective amount” of an ER ligand is any amount of one or more ER ligands or combinations thereof that cause an increase in cell proliferation or differentiation into the desired tissue type. Typically this amount will be between about 10⁻⁴ and about 10⁻¹⁴ M of the ER ligand, more preferably between about 10⁻⁶ and about 10⁻¹² M, more preferably between about 10⁻⁸ and 10⁻¹¹ M or 10⁻⁶ and about 10⁻⁹ M. Depending on the ER ligand, adult stem cell, and desired tissue type, a lower concentration of ER ligand may be preferably over a higher concentration.

For cell proliferation, the adult stem cells are incubated for at least about 4 days with a medium comprising an effective amount of an ER ligand. The medium can be any growth medium known in the art used to grow and maintain adult stem cells. Supplementing the growth medium with an effective amount of one or more ER ligands increases the number of adult stem cells compared to the same conditions absent the ER ligand and has little to no adverse effect on the differentiating potential of the cells. In another embodiment, the adult stem cells are incubated for at least about 7 days with a medium comprising an effective amount of an ER ligand. In another embodiment, the adult stem cells are incubated for at least about 10 to about 14 days with a medium comprising an effective amount of an ER ligand.

After incubating the adult stem cells with a growth medium supplemented with one or more ER ligands, the growth medium is removed and the cells are washed. To proceed with differentiation, the washed cells are contacted with a solution used to determine differentiation, such as an osteogenic medium or adipogeneic medium, supplemented with an effective amount of one or more ER ligands. The one or more ER ligands can be the same ligands used during the proliferation stage or other ER ligands. The effective amount of ER ligands used during the proliferation stage may be the same or different as the effective amount used during the differentiation stage. Additionally, the amount of ER ligand used in either stage may be optimized or varied depending on whether the cells came from a male member of the species as opposed to a female member of the species.

### EXAMPLES

The examples set forth below illustrate but are not meant to limit the invention.

### Example 1

### Estradiol Enhancing Osteogenic Differentiation of Human Mesenchymal Stromal Cells And Adipose-Derived Stromal Cells

### Isolation And Differentiation of Human MSCs And ASCs

Human bone marrow MSCs were provided by the Center for Gene Therapy of Tulane University. The MSCs were isolated from bone marrow cells donated by a healthy adult male volunteer. The passage-1 cells were recovered and expanded by culture in a basic medium composed of DMEM, 10% FBS and 1% antibiotic-antimycotic. Human ASCs were isolated from a 45 year old, healthy female with a lipo-aspirated surgery. The abdomen subcutaneous adipose tissue was finely minced and then digested with 0.075 wt % collagenase type I at 37° C. for 45 min. The cells were then collected by filtration with a cell strainer with pore size of 100 μm diameter. The ASCs were expanded in basic DMEM medium. Upon reaching 80-90% confluence of human MSCs and ASCs, the cells were trypsinized, counted, and subcultured at a density of 105 cells/100 mm dish. The cells used for study were passage-2. For osteogenic differentiation, the cells were seeded at a cell density of 104 cells/cm2 on 12-well plates and 100-mm petri dishes. Upon reaching 70% confluence, counting as day-0, the cells were exposed to osteogenic differentiation medium supplemented with either 10 nM or 10 pM E2. The osteogenic differentiation medium consisted of basic medium supplemented with 100 nM dexamethasone, 10 mM β-glycerophosphate, and 0.05 mM ascorbic acid-2-phosphate. At the defined time, the cells were collected and analyzed for osteogenic gene expression and differentiation potentials.

### Quantitative Assessment of Osteogenic Potentials

The cultured cells in 12-well plates were washed twice with PBS, suspended and lysed in 1% Triton-X100 solution. The collected cells were subsequently homogenized using sonication. The DNA content was determined by a fluorometric assay (Bio-Rad). ALP and calcium content were measured by spectrophotometry with a calorimetric kit according to the manufacturers' instructions (Sigma).

### Analysis of Osteogenic Gene Expression

RNA was extracted from differentiated cells using TRizol reagent (Invitrogen). 5 μg of total RNA was converted to single stranded cDNA using a commercial cDNA synthesis kit. Aliquots of the cDNA were amplified with Ampli-Taq DNA polymerase for ALP, osteocalcin (OCN), core binding factor alpha1 (cbfa1), peroxisome proliferator activated receptors gamma (PPARγ)-2, estrogen receptors (ER) alpha (α) and beta (β), and housekeeping gene GAPDH. 40 cycles were used, each consisting of 45 sec of denaturation at 95° C., 45 sec of annealing at 58° C., and 1 min of polymerization at 72° C.

### Results

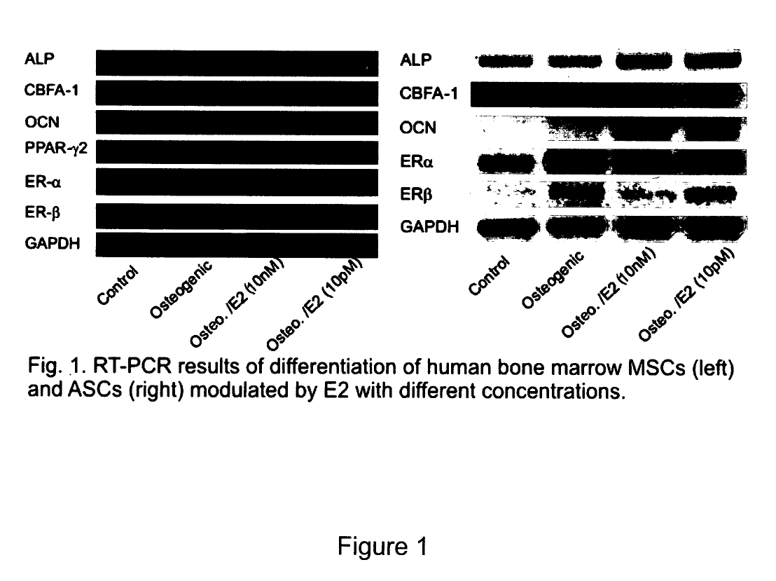

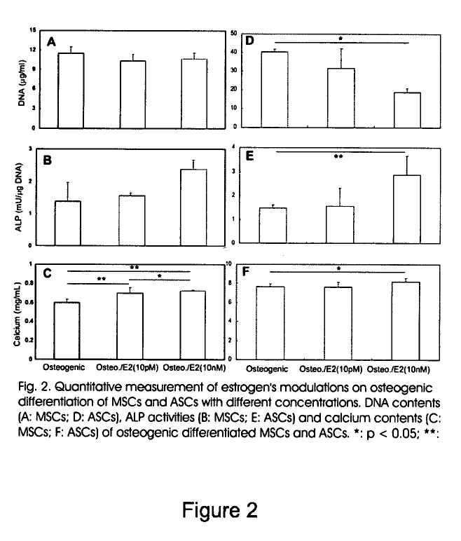

After exposure to an osteogenic medium, human bone marrow MSCs and ASCs condensed and formed high cell-density nodules. These nodules were positively stained by ALP and von Kossa. Few mineralized nodules were observed in MSCs exposed to basic medium. The denser color of ALP and von Kossa staining were observed at MSCs and ASCs stimulated by the differentiation medium supplemented with E2. On day 7, osteogenic gene expressions of MSCs and ASCs were identified by RT-PCR (FIG. 1). The specific markers of early osteogenic differentiation, ALP and cbfa-1 were expressed in all of the differentiation media supplemented with or without estrogen. However, the estrogen-stimulated osteogenic differentiation exhibited a denser band of cbfa-1 than that of differentiation without estrogen supplement. Furthermore, OCN, a bone turnover marker, was only densely expressed in the MSCs and ASCs cultured with E2 supplements at a concentration of 10 nM. PPAR-γ2, a specific marker for adipogenesis, was expressed in MSCs treated with the standard osteogenic medium, but was inhibited by estrogen supplements. ER α, instead of ER β, is up-regulated in MSCs and ASCs exposed to osteogenic medium and the medium with E2. Quantitatively, a higher dose of E2 supplement inhibits cell proliferation (measured by the amount of DNA) of ASCs during osteogenesis (FIG. 2D) but does not affect MSCs proliferation during osteogenesis (FIG. 2A). The ALP activities of differentiated ASCs with estrogen supplement were significantly improved after 14 days (FIG. 2E), however, little statistical significance was observed in MSCs (FIG. 2B). The calcium content of both differentiated MSCs and ASCs are significantly increased by addition of estrogen (FIGS. 2C and 2F).

### Discussion

Estrogen has exhibited regulative roles in multiple differentiations of different cells. Specifically with regard to embryos, estradiol was reported to improve osteogenic and adipogenic differentiations of bone marrow MSCs and neurogenic differentiation of embryonic stem cells (Murashov et al. (2004) FEBS Lett. 569:165-8). Estradiol was shown to exert its osteogenic function in bone formation via release or up-regulation of a number of cytokines (IL1 and 6), prostaglandin E2 and osteogenic growth factors (BMPs, TGF-β1 and IGF) (Zhou et al. (2003) Mol Endocrinol. 17:56-66; Joo et al. (2004) Arch Pharm Res. 27:99-105). Recent studies show that estrogen up-regulates the expression of osteogenic growth factors (BMPs and TGF-β1) of osteoblasts and bone MSCs for human and mice in vitro (Bilezikian A, et al (2002) Principles of Bone Biology). These cytokine, hormone and osteogenic growth factors can further promote proliferation and differentiation of osteo-progenitor cells through autocrine or paracrine mechanisms. Estrogen also modulates synthesis of bone matrix protein (Cao et al. (2003) J Cell Biochem. 89:152-164). These characteristics of estrogen properly explained the functions of E2 on regulating osteogenic differentiation of human MSCs and ASCs in the present study. Based on the preliminary data, an optimal utilization of estrogen can effectively improve the differentiations of MSCs and ASCs that may serve as cell sources for bone tissue engineering.

### Example 2

### 17-β Estradiol Enhances Osteogenic And Adipogenic Differentiations of Human Adipose-Derived Stromal Cells

### Isolation And Expansion of Human ASCs

Because estrogen receptors (ERs) exist in adipose tissues (Dieudonne et al. (2004) Am J Physiol Cell Physiol. 286:C655-661), it is hypothesized that estrogen can also modulate the differentiation potentials of ASCs. All the experiments using human ASCs were performed under an approved IRB protocol of the University of Illinois at Chicago. Human abdomen subcutaneous adipose tissue was obtained from a 45 year old, healthy female with lipo-aspirated surgery. ASCs were isolated from adipose tissue according to previous studies (Hong et al. (2006) Cells Tissues Organs. 183 (3):133-40). Briefly, adipose tissue was finely minced and then digested with 0.075 wt % collagenase type I (Sigma, St Louis, Mo.) at 37° C. for 45 min with intermittent shaking. The cells were collected by filtration with a cell strainer with pore size of 100 μm diameter (Fisher Scientific, Barrington, Ill.). The harvested cells were cultured in a Dulbecco's Modified Eagle's Medium (DMEM), 15% fetal bovine serum (FBS) and 1% antibiotic-antimycotic at 106 cells/100 mm dish. Upon reaching 80-90% confluence, the isolated cells were trypsinized, counted, and subcultured at a density of 10⁵ cells/100 mm dish as passage-1. The cells used for study are passage-3.

### Osteogenic Differentiation of ASCs Modulated By E2

The basic medium supplemented with 100 nM dexamethasone, 10 mM β-glycerophosphate, and 0.05 mM ascorbic acid-2-phosphate was used to differentiate ASCs into an osteogenic cell lineage. Human ASCs were seeded at a cell density of 10⁴ cells/cm² on 12-well plates and 100 mm petri dishes. Upon reaching 70% confluence, counting as day-0, the cells were exposed to osteogenic differentiation medium supplemented with different concentrations of E2 (from 10⁻⁸ to 10⁻¹¹ M). On day 7, the cells cultured on petri dishes were collected for assessment of gene expressions of osteogenic specific markers and estrogen receptors (ERs) by reverse transcriptase polymerase chain reaction (RT-PCR) analysis. On day 14, the cells cultured in 12-well plates were analyzed for DNA, extracellular matrix, alkaline phosphatase (ALP) activity and calcium content.

### Adipogenic Differentiation of ASCs Modulated By E2

The basic DMEM medium supplemented with 50 nM of dexamethasone, 10 mM of insulin and 5 mM of isobutyl-methylxanthine (IBMX) was used to differentiate ASCs into adipocytes (Hong et al. (2005) Ann Biomed Eng. 33:511-517). At 70% confluence of ASCs in 12-well plates, the basic DMEM medium was exchanged for the adipogenic differentiation medium. The effects of estrogen on adipogenic differentiation were observed after E2 supplements at concentrations ranging from 10⁻⁸ to 10⁻¹¹M. On day 4, the cells were collected for analysis of adipogenic and ER gene expressions. On day 7, adipogenic differentiation of ASCs modulated by estrogen was analyzed by Oil-O-Red staining and quantitative measurements. Each treatment group included four samples.

### Quantitative Measurements of DNA Content And Protein

On the defined day, cells cultured in 12-well plates were washed twice with phosphate buffered solution (PBS), suspended and lysed in 1% Triton-X100 solution (Sigma, St. Louis, Mo.). The collected cells were subsequently homogenized using sonication (Dismembrator Model 100, Fisher Scientific, Barrington, Ill.). The DNA content in the cells was determined by a fluorometric assay using a spectrofluorometer and DNA quantification kit (Hoechst 33258, Bio-Rad, Hercules, Calif.). The total protein concentrations of lysates were measured by a simple colorimetric assay based on the Bradford dye-binding procedure (Bio-Rad, Hercules, Calif.). The amount of protein was normalized by the unit of DNA.

### Assessment of ALP Activity And Mineralization

ALP activity was assessed qualitatively by ALP staining. The cultured cells were fixed in 10% paraformaldehyde and incubated with 120 mM Tris buffer, pH 8.4, containing 0.9 mM Naphtol AS-MX Phosphate (Sigma, St. Louis, Mo.) and 1.8 mM Fast Red TR. After 45 min at room temperature, the cultures were washed with deionized water. Mineral deposition in the extracellular matrix was presented by von Kossa staining. The fixed cells were incubated in 2.5% (w/v) silver nitrate (Sigma, St. Louis, Mo.) solution for 30 min in sunlight. In order to quantify ALP activity and calcium content, cultured cells were washed twice with PBS, suspended and lysed in 0.5 ml of 1% Triton-X100 solution. ALP and calcium content were measured by spectrophotometry with a colorimetric kit according to the manufacturer's instructions (Raichem, San Diego, Calif.).

### Oil-O-Red Staining And Quantitative Measurement of Adipogenesis

After 7 days of incubation with adipogenic differentiation medium supplemented with different concentrations of E2, cultured cells were fixed in 10% neutral buffered formalin for 15 min and washed with PBS twice. Cells were immersed in a 60% Oil-O-Red solution in PBS (Sigma, St Louis, Mo.) for 60 min. In order to quantify adipogenic potentials, the images of Oil-O-Red stained at each well were randomly taken by a digital camera connected to a computer monitor. The Oil-O-Red positive cells on the screen were double-blindly counted. Five pictures were taken from each well. The cell numbers were normalized to per mm². Oil-O-Red dye then was eluted by 500 μl of 100% isopropanol for each well of cultured cells. The spectrophotometric absorbance of the resulting solution was quantified at 510 nm.

### RT-PCR Analysis

Total RNA was extracted from the various differentiated cells using TRizol reagent (Invitrogen, Carlsbad, Calif.). Approximately 5 μg of total RNA was converted to single stranded cDNA using a commercial cDNA synthesis kit (Invitrogen, Carlsbad, Calif.). Aliquots of the cDNA were amplified with Ampli-Taq DNA polymerase (PerkinElmer, Norwalk, Conn.) for ALP, osteocalcin (OCN), core binding factor alphal (cbfa1), peroxisome proliferator activated receptors gamma (PPARγ)-2, lipoprotein lipase (LPL), estrogen receptors alpha (ERα) and beta (ERβ). Forty cycles were used for all PCR experiments, each consisting of 45 sec of denaturation at 95° C., 45 sec of annealing at 62° C., and 1 min of polymerization at 72° C., followed by a final 10 min extension at 72° C. The housekeeping gene glyceraldehyde-3-phosphate dehydrogenase (GAPDH) was used as a control for RNA loading samples. PCR products were analyzed electrophoretically with an Agilent 2100 bioanalyzer (Agilent Technologies, Palo Alto, Calif.).

### Statistical Analysis

All quantitative data were expressed as mean±standard deviation. The osteogenic and adipogenic potentials of each group of different treatments were analyzed by one-way ANOVA with the use of commercially available statistical software (SPSS). Turkey's post hoc comparison was used to determine statistical differences between treatment groups; p values less than 0.05 were considered significant.

### Results—E2 Enhances Osteogenesis of Human ASCs

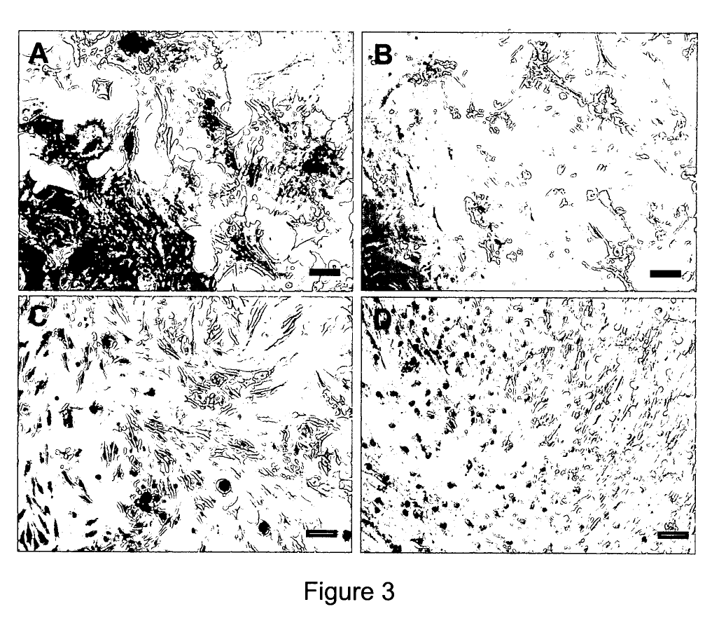

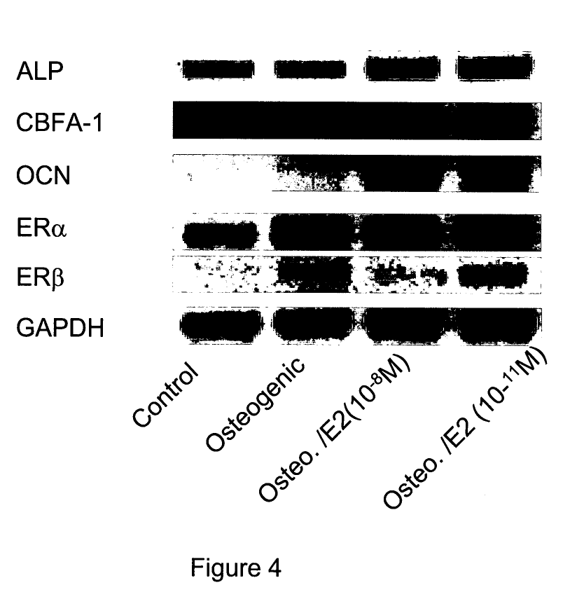

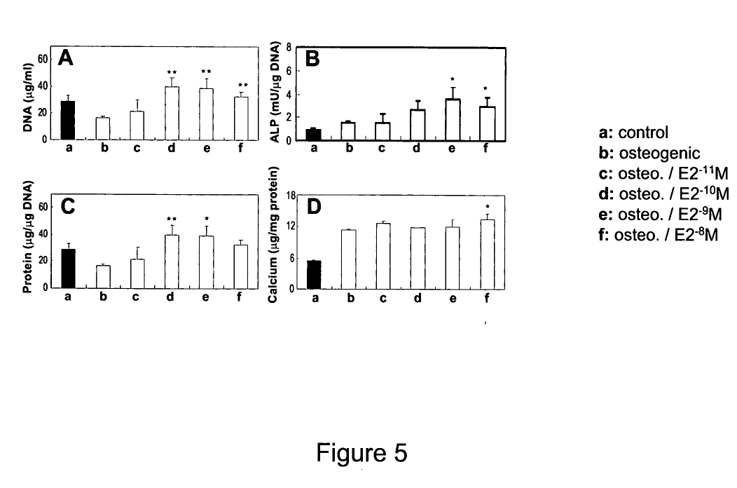

After exposure to osteogenic medium, cells became condensed and formed high cell-density nodules positively stained by ALP and von Kossa (FIG. 3A). In contrast, few ALP and von Kossa positively stained nodules were observed in the cells exposed to basic medium (FIG. 3B). Intense staining for ALP and von Kossa were observed in ASCs stimulated by the medium supplemented with E2. FIG. 4 reveals osteogenic gene expressions of human ASCs after 7 days of exposure to differentiation medium supplemented with different concentrations of E2 (FIG. 4). The early osteogenic differentiation specific markers, ALP and cbfa-1, were expressed in all of the differentiation media with or without estrogen supplement. However, the osteogenic differentiation stimulated by estrogen supplement exhibited intense bands for ALP and cbfa-1 than those cells grown in the absence of estrogen. Furthermore, OCN, a bone turnover and differentiated osteoblast marker, was only expressed in the ASCs cultured with estrogen supplement at a concentration of 10⁻⁸ M. ER α was expressed in ASCs exposed to osteogenic medium and the medium with estrogen supplement. Only the cells exposed to osteogenic media and with estrogen supplement of 10⁻¹¹M expressed light bands for ER β. Quantitatively, the population of ASCs indicated by the amount of DNA content varied with the concentrations of estrogen supplement (FIG. 5A). This indicates that the osteogenic medium with or without estrogen supplement does not increase proliferation during osteogenesis. The ALP activities observed in differentiated ASCs with E2 supplement at concentrations of 10⁻⁹ and 10⁻⁸ M significantly improved after 14 days (FIG. 5B). The protein content of the differentiated ASCs was increased by E2 supplement, indicating estrogen's capability of improving extracellular matrix synthesis (FIG. 5C). The mineralization of the extracellular matrix, indicated by calcium content, significantly increased by estrogen supplement most notably at a concentration of 10⁻⁸ M (FIG. 5D).

### E2 Enhances Adipogenic Differentiation of Human ASCs

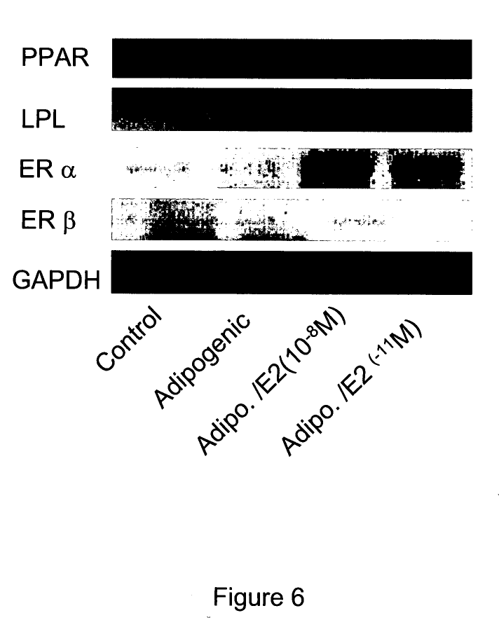

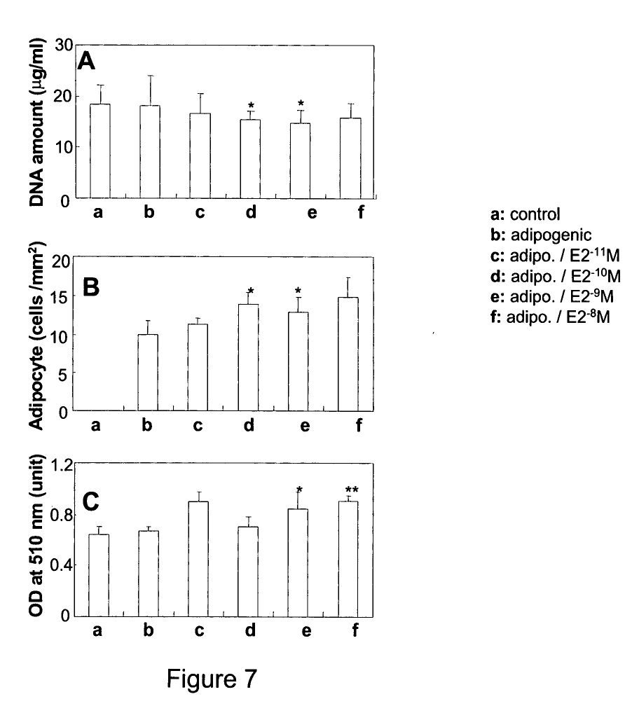

Adipogenic differentiation medium effectively turns ASCs into adipocyte lineages, characterized by lipid accumulation (FIG. 3C). No lipid accumulation was found in ASCs exposed to basic medium (FIG. 3D). An increased population of lipid-containing cells was found in ASCs after E2 supplement. After 4 days of differentiation, PPARγ-2 and LPL were up-regulated and expressed in the ASCs exposed to adipogenic differentiation medium and the cells exposed to the differentiation medium with different concentrations of estrogen supplement (FIG. 6). ERα was expressed in ASCs exposed to the differentiation medium with E2 supplement, while no ERα band was found after adipogenic differentiation without E2 supplement. ERβ was undetected in all cells with adipogenic differentiation (FIG. 6). Quantitatively, the DNA amount of ASCs exposed to adipogenic media decreased following the addition of E2 supplements, indicating that E2 supplement does not affect cell proliferation during adipogenesis (FIG. 7A). The number of Oil-O-Red positively stained cells was increased in the adipogenic differentiated group with estrogen supplement (FIG. 7B). The elution of Oil-O-Red staining of differentiated ASCs increased 25.5% and 33.9% with estrogen supplements at concentrations of 10⁻⁹ and 10⁻⁸ M, respectively, which was significantly higher than the differentiation group without the estrogen supplement (FIG. 7C). However, the effects of estrogen supplement at low concentrations on adipogenesis were hardly observed.

### Discussion

This study demonstrates the effects of estrogen modulation on human ASC's differentiation potential into osteogenic and adipogenic pathways. After exposure to osteogenic and adipogenic differentiation media supplemented with different concentrations of E2, human ASCs demonstrated improved adipogenic and osteogenic capabilities. ASCs treated with osteogenic medium with E2 supplement showed up-regulation of cbfa-1, OCN gene expression, enhanced ALP activity, extracellular matrix synthesis and mineralization of osteogenic differentiated ASCs. In addition, E2 effectively increased adipocyte cell population and lipid accumulation in ASCs treated with adipogenic medium. These results demonstrate that estrogen can effectively improve differentiation potentials of multipotent ASCs in tissue engineering and regenerative medicine.

Being similar to bone marrow MSCs, ASCs can be differentiated into multiple lineages of both mesodermal and ectodermal cells including osteoblastic, chondrocytic, adipocytic and neurogenic cells. ASC-based tissue engineering and regeneration is dependent upon the ability of ASCs to differentiate into specific lineages. In addition, the capability of ASC osteogenic and chondrogenic differentiation potential is lower than MSCs isolated from bone marrow. Optimization of in vivo tissue regeneration requires effective ASC modulation in vitro to improve their differentiation potentials.

Similar to studies of improved MSC differentiation through E2 supplement, this example demonstrates that E2 improved osteogenic and adipogenic differentiation in human ASCs. Quantitatively measured osteogenic potentials of differentiated ASCs, including ALP activity, extracellular matrix synthesis, and mineralization, significantly improved. The osteogenic-specific gene expressions were all up-regulated by E2 supplement. Specifically, OCN, a marker for terminally differentiated osteoblast, was only expressed in osteogenic medium supplemented with a high concentration of E2. In addition, adipogenic differentiation of ASCs, including adipocyte number and lipid accumulation, are significantly increased with an E2 supplement. The dose-dependent function of E2 and its mechanism via ER on ASCs are similar to bone MSCs. Taken together, the results demonstrate that E2 can have a positive regulation on osteogenic and adipogenic differentiation potentials in human ASCs.

### Example 3

### Estrogen Enhancement of the Proliferation of Human Mesenchymal Cells

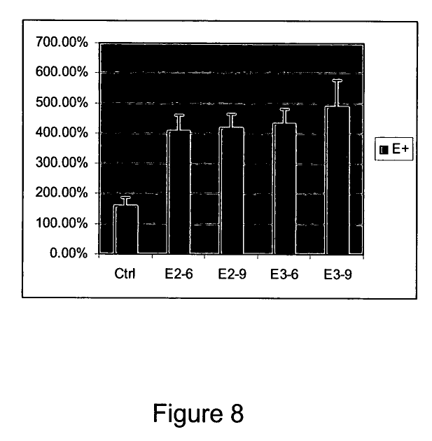

Previous studies suggest that estrogen enhancement does not increase proliferation of adult stem cells during osteogenesis or adipogenesis. This study investigates the mitogenic effects of estrogen receptor ligands on human bone marrow MSCs that is not contacted with osteogenic or adipogenic medium. Purified human bone marrow mesenchymal stem cells (MSCs) were purchased commercially. MSCs were respectively exposed to basic medium (DMEM+10% FBS+1% antibiotics), and basic medium supplement with 10⁻⁶ and 10⁻⁹ M 17-β estradiol (E2) and estriol (E3) for approximately one week. Then, various cells were plated into 96-well plates in 500 cells/well and continued to be exposed to the media. After 4 days, cell densities were measured by MTS methods and compared to initial densities. FIG. 8 illustrates the increase of the number of MSCs after being treated with basic medium and basic medium supplemented with E2 or E3.

MTS assay and cell counting revealed that the cell proliferation is greatly increased in the MSCs modulated by both E2 and E3, demonstrating that estrogen receptor ligands can significantly improve the proliferation of human MSCs. Furthermore, estrogen modulation did not affect the differentiation capabilities of the human MSCs. The estrogen-modulated MSCs possessed the same adipogenic and osteogenic differentiation potentials as the controls. This preliminary study shows that E2 and E3 can effectively improve the growth of human bone marrow MSCs and maintain their differentiation capabilities, indicating their potential to provide the requisite quantity of adult stem cells, particularly MSCs, for tissue engineering.

### Example 4

### Gender-Derived Differences of Estrodial Modulation On Bone Marrow MSC-Based Bone Tissue Engineering

This study compared the optimal concentration of ER ligands for regulatory function of proliferation and osteogenic differentiation between genders.

### Isolation And Proliferation of Rat Bone MSCs

Three month-old, male and female Fisher 344 rats (Charles River Lab., Wilmington, Mass.) were purchased and euthanized by CO² inhalation. The tibias and femurs were dissected and bone marrow plugs were flushed out with a syringe (18-gauge needle), and filled with a basic medium consisting of DMEM, 10%FBS and 1% antibiotic-antimycotic. Cells were counted on a hemacytometer, plated at 5×10⁷ cells/100-mm petri dish and incubated with basic DMEM medium at 37° C. in 5% CO₂. The MSCs were isolated by removing unattached cells via medium exchange. The MSCs were incubated up to 80% confluence and then subcultured at a density of 10⁶/100-mm dish. Prior to confluence, the MSCs were trypsinized, counted and used for measuring proliferation rate using E2 modulation.

In order to analyze the estrogen function on cell proliferation, 500 of passage-1 MSCs were placed into each well of 96-well plates and exposed to basic medium supplemented with different concentrations of E2 (10⁻⁶M to 10⁻⁹M) (Sigma, St. Louis, Mo.). On day 3, the cell proliferation with different estrogen modulations were quantified using a MTS calorimetric method according to manufacturer's manual (CellTiter 96® Queous One Solution Cell Proliferation Assay, Promega, Madison, Wis.)

### Preparation And Osteogenic Differentiation of TE Constructs

TE constructs were created by seeding passage-1 MSCs on gelatin sponges (Gelfoam@, Pharmacia & Upjohn, Kalamazoo, Mich.). The sponge were trimmed into 4×4×4 mm³ and pre-wet with DMEM medium for 1 hr. The sponges were transferred to MSC suspension with a cell density of 2×10⁶ cells/ml under a slight vacuum created by 20 ml syringe, followed by incubation in 5% CO₂ at 37° C. for two hours. Beginning the next day as day-0, TE constructs were respectively exposed to the osteogenic differentiation medium supplemented with different concentrations of E2 (10⁻⁶M to 10⁻¹²M). The osteogenic differentiation medium consists of basic medium supplemented with 100 nM dexamethasone, 10 mM β-glycerophosphate, and 0.05 mM ascorbic acid-2-phosphate. After two weeks of differentiation, constructs of each experimental group were collected and quantitatively measured for osteogenic differentiation.

### Quantitative Assessment of Osteogenic Potentials

TE constructs were immersed in 0.2 ml of 1% Triton-X100 and homogenized with sonication. The DNA concentration of cell lysate was measured fluorometrically using Hoechst dye 33258 (Bio-Rad, Hercules, Calif.). The fluorescent optical density of each sample was measured using a fluorometer having an excitation wavelength of 360 nm and an emission wavelength of 460 nm. The amount of DNA in each sample was determined by using a prepared standard curve. ALP and calcium content were measured by spectrophotometry with a calorimetric kit according to the manufacturers' instructions (Sigma, St. Louis, Mo.).

### Results

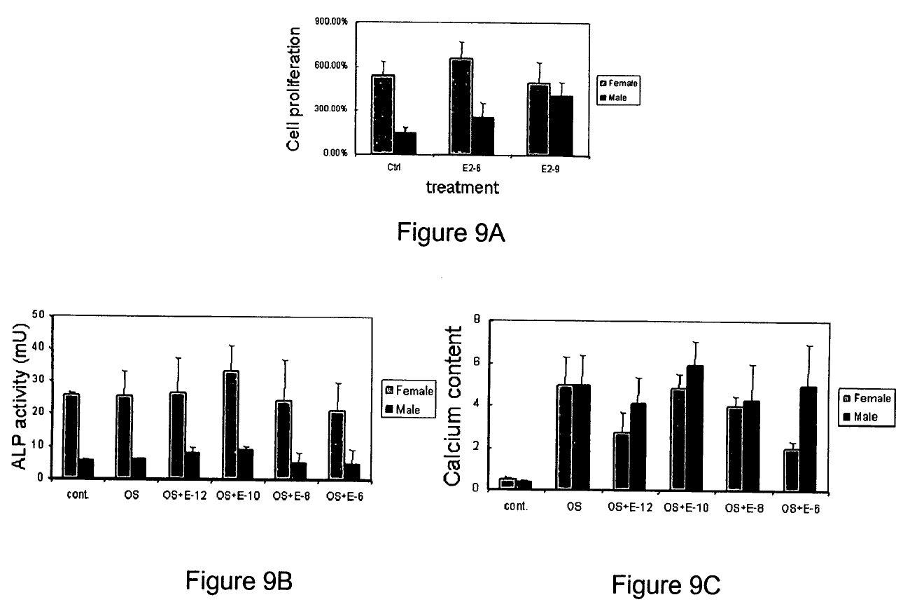

After 3 days, the effects of E2 modulation on cell proliferation of bone MSCs were demonstrated by CellTiter 96® Aqueous One Solution Cell Proliferation Assay (FIG. 9A). With basic medium as well as medium supplemented with E2, female bone MSCs proliferated much faster than male bone MSCs. Absorbance at 540 nm of MSCs cultured with basic medium supplement of 10⁻⁹ and 10⁻⁶ M E2 showed significantly higher cell proliferation than the control for both male and female groups. Interestingly, the male bone MSCs proliferation peak is at 10⁻⁹ M of E2 whereas female's peak is at 10⁻⁶ M. This indicates that E2 can effectively improve the proliferation of rat bone MSCs on both male and female, however, optimal concentrations is varied with the genders.

After exposure to osteogenic differentiation medium, rat bone MSC-based TE constructs exhibited osteogenic potentials confirmed by ALP and von Kossa staining. Quantitatively, after 14 days differentiation, osteogenic potentials of differentiated constructs in terms of ALP activities (FIG. 9B) and calcium contents (FIG. 9C) of both male and female MSCs were increased. ALP activities of TE constructs created by female bone MSCs are higher than those of male's. The presence of E2 increased ALP activities of both male and female constructs in a dose-dependent manner. The peak concentration of E2 to increase ALP activity is 10⁻¹⁰ M for both male and female MSCs. E2 may also improve calcium deposition in male MSC-derived constructs but does not appear to have similar effects on female's MSCs.

Estrogen exerts its osteogenic function via release or up-regulation of a number of cytokines (IL1 and 6), prostaglandin E2 and osteogenic growth factors (BMPs, TGF-β1 and IGF). Therefore, estrogen can be utilized to improve efficiency of autologous bone marrow MSCs for bone tissue engineering. The present experiment demonstrates that the regulation of estrogen on bone marrow stem cell proliferation and osteogenic differentiation are dose-dependent and that the optimal concentrations are varied with genders.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

One of ordinary skill in the art will appreciate that starting materials, reagents, purification methods, materials, substrates, device elements, analytical methods, assay methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim.

All references cited herein are hereby incorporated by reference in their entirety to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional nucleic acids, chemically modified nucleic acids, additional cells, and additional uses of the invention. All headings used herein are for convenience only. All patents and publications mentioned in the specification are indicative of the levels of skill of those skilled in the art to which the invention pertains, and are herein incorporated by reference to the same extent as if each individual publication, patent or patent application was specifically and individually indicated to be incorporated by reference. References cited herein are incorporated by reference herein in their entirety to indicate the state of the art as of their publication or filing date and it is intended that this information can be employed herein, if needed, to exclude specific embodiments that are in the prior art.

1. A method of enhancing the differentiation of adult stem cells comprising the steps of:
a) isolating said adult stem cells;
b) contacting said isolated cells with a solution to determine differentiation; and
c) contacting said adult stem cells with a first effective amount of one or more estrogen receptor ligands.

2. The method of claim 1 wherein said adult stem cells are bone marrow mesenchymal stem cells.

3. The method of claim 2 wherein said solution to determine differentiation is an osteogenic medium.

4. The method of claim 2 wherein said solution to determine differentiation is an adipogenic medium.

5. The method of claim 1 wherein said adult stem cells are adipose-derived stromal cells.

6. The method of claim 5 wherein said solution to determine differentiation is an osteogenic medium.

7. The method of claim 5 wherein said solution to determine differentiation is an adipogenic medium.

8. The method of claim 1 wherein said first effective amount is between about 10⁻⁶ and about 10⁻¹² M of said one or more estrogen receptor ligands.

9. The method of claim 1 wherein said first effective amount is between about 10⁻⁸ and about 10⁻¹¹ M of said one or more estrogen receptor ligands.

10. The method of claim 1 wherein said first effective amount is between about 10⁻⁶ and about 10⁻⁹ M of said one or more estrogen receptor ligands.

11. The method of claim 1 wherein said first effective amount is adjusted depending on the gender of the cells.

12. The method of claim 1 wherein said one or more estrogen receptor ligands are selected from the group consisting of estriol, estradiol, estrone and combinations thereof.

13. The method of claim 1 wherein the estrogen receptor ligand is 17-β estradiol.

14. The method of claim 1 further comprising increasing the proliferation of said isolated cells by contacting said isolated cells with a second effective amount of one or more estrogen receptor ligands prior to contacting said cells with the solution to determine differentiation.

15. The method of claim 14 wherein said second effective amount is between about 10⁻⁶ and about 10⁻⁹ M of said one or more estrogen receptor ligands.

16. The method of claim 14 wherein said one or more estrogen receptor ligands comprise estriol, 17-β estradiol, or both.

17. The method of claim 14 wherein said second effective amount is adjusted depending on the gender of the cells.

18. The method of claim 17 wherein said second effective amount is approximately 10⁻⁹ M of said one or more estrogen receptor ligands where the cells are male.

19. The method of claim 17 wherein said second effective amount is approximately 10⁻⁶ M of said one or more estrogen receptor ligands where the cells are female.
